(19) United States
(12) Patent Application Publication
(10) Pub. No.: US 2011/0206756 A1
(43) Pub. Date: Aug. 25, 2011

(54) DITHIOCARBAMATE METAL CHELATES AND METHODS OF MAKING AND USING THEREOF

(75) Inventors: Paul J. Shami, Sandy, UT (US); Ken M. Kosak, West Valley City, UT (US); Thomas Kennedy, Charlotte, NC (US)

(73) Assignee: UNIVERSITY OF UTAH RESEARCH FOUNDATION, Salt Lake City, UT (US)

(21) Appl. No.: 12/922,904

(22) PCT Filed: Mar. 16, 2009
(86) PCT No.: PCT/US2009/037216

§ 371 Date: May 13, 2011

Related U.S. Application Data
(60) Provisional application No. 61/037,095, filed on Mar. 17, 2008.

(51) Int. Cl. A61K 9/127; A61K 39/395; A61K 31/175; A61K 31/56; A61K 33/36; A61K 31/07; A61K 47/24; A61P 35/00; A61K 47/26; A61K 47/44

(52) U.S. Cl. 424/450; 424/141.1; 514/589; 514/169; 424/623; 514/725; 514/788; 514/784; 514/777

(57) ABSTRACT

Described herein are compositions useful in anticancer treatment and prevention. The compositions are composed of (a) a dithiocarbamate metal chelate and (b) an amphiphile, wherein the amount of amphiphile is sufficient to produce a liposome or micelle. Methods for using the compositions in anticancer treatment and prevention are also described herein.

### CROSS REFERENCE TO RELATED APPLICATIONS

This application claims priority upon U.S. provisional application Ser. No. 61/037,095, filed Mar. 17, 2008. This application is hereby incorporated by reference in its entirety for all of its teachings.

### ACKNOWLEDGEMENTS

The research leading to this invention was funded in part by the National Institutes of Health grant number RO1 CA129611. The U.S. Government may have certain rights in this invention.

### BACKGROUND

Cancer, the uncontrolled growth of malignant cells, is a major health problem of the modern medical era and ranks second only to heart disease as a cause of death in the United States. For example, Acute Myelogenous Leukemia (“AML”) is the most common acute leukemia in adults (Greer, J. P., et al. (2004) Wintrobe's Clinical Hematology, Baltimore pp. 2098-2142). Most patients who contract this disease succumb to it. Since the early seventies, the mainstay of therapy has been cytosine arabinoside (Ara-C) and anthracyclines (Greer, J. P., et al. (2004) Wintrobe's Clinical Hematology, Baltimore pp. 2098-2142). With the notable exception of all-trans retinoic acid for the treatment of acute promyelocytic leukemia, no new agent has had a major impact on disease outcome. Stem cell transplantation for AML has its limitations due to patients' age and availability of suitable donors. Furthermore, recent trials suggest that high dose therapy with stem cell rescue may not offer a survival advantage over standard dose chemotherapy (Cassileth, P. A., et al. (1998) New England Journal of Medicine 339(23): 1649-1656). Consequently, the need for new agents with new mechanisms of action for the treatment of AML and other types of cancer is evident.

### SUMMARY

Described herein are compositions useful in anticancer treatment and prevention. The compositions are composed of (a) a dithiocarbamate metal chelate and (b) an amphiphile, wherein the amount of amphiphile is sufficient to produce a liposome or micelle. Methods for using the compositions in anticancer treatment and prevention are also described herein. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows the effect of DETC/metal chelates on HL-60 cell growth in vitro.

FIG. 2 illustrates leukemia cell apoptosis induction by DETC/Cu in HL-60 cells cultures with 0.32 μM DETC/Cu chelate for 24 hours upon which the number of apoptotic cells was measured by flow cytometry using PI staining.

FIG. 3 shows the effect of DETC/Cu chelate on leukemia cell growth in vivo.

FIG. 4 shows the effect of DETC/Cu²⁺ chelates on the growth of 3 solid tumor cell lines in vitro.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or cannot be substituted and that the description includes both unsubstituted lower alkyl and lower alkyl where there is substitution.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound. A weight percent of a component, unless specifically stated to the contrary, is based on the total weight of the formulation or composition in which the component is included.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or a maximum. Furthermore, such an interpretation should apply regardless of the breadth of the range or the characteristics being described.

The term “alkyl group” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like.

The term “alkenyl group” as used herein is a branched or unbranched alkyl group of 2 to 24 carbon atoms possessing at least on C═C group. The alkenyl group can be substituted with one or more groups including, but not limited to, halo, hydroxy, alkylthio, arylthio, alkoxy, aryloxy, amino, mono- or di-substituted amino, ammonio or substituted ammonio, nitroso, cyano, sulfonato, mercapto, nitro, oxo, cycloalkyl, benzyl, phenyl, substituted benzyl, substituted phenyl, benzylcarbonyl, phenylcarbonyl, saccharides, substituted benzylcarbonyl, substituted phenylcarbonyl and phosphorus derivatives.

The term “alkoxy group” as used herein is represented by the formula —OR, where R is an alkyl group as defined herein.

The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, halo, hydroxy, alkylthio, arylthio, alkoxy, aryloxy, amino, mono- or di-substituted amino, ammonio or substituted ammonio, nitroso, cyano, sulfonato, mercapto, nitro, oxo, alkyl, alkenyl, cycloalkyl, benzyl, phenyl, substituted benzyl, substituted phenyl, benzylcarbonyl, phenylcarbonyl, saccharides, substituted benzylcarbonyl, substituted phenylcarbonyl and phosphorus derivatives. The aryl group can include two or more fused rings, where at least one of the rings is an aromatic ring. Examples include naphthalene, anthracene, and other fused aromatic compounds.

The term “reduce” refers to lowering the rate of cancer cell growth or tumor growth. For example, the cancer cell growth rate can be reduced by 5%, 10%, 20%, 30%, 40%, 50%, 60%, 70%, 80%, 90%, or 99% when compared to a positive control.

The term “prevent” refers to zero cancer cell growth rate or tumor growth when compared to a positive control.

The term “micelle” refers an aggregate of amphiphilic molecules dispersed in a liquid colloid. A typical micelle in aqueous solution forms an aggregate with the hydrophilic “head” regions in contact with surrounding medium, sequestering the hydrophobic tail regions in the micelle center. The shape and size of a micelle is a function of the molecular geometry of the amphiphiles and solution conditions such as amphiphile concentration, temperature, pH, and ionic strength. The dithiocarbamate metal chelate is for the most part incorporated within the hydrophobic portion of the micelle.

The term “liposome” refers to a bilayered system produced by an amphiphile. An aqueous core is present in the liposome as a result of the hydrophobic tails of the amphiphile lining up to produce the bilayer.

Variables such as An, R¹-R⁴, a, b, and n used throughout the application are the same variables as previously defined unless stated to the contrary.

### I. Compositions and Preparation Thereof

Described herein are pharmaceutical compositions useful for anticancer treatment. The compositions described herein are composed of a dithiocarbamate metal chelate in liposomes or micelles that can be readily administered to a subject for the treatment of cancer. The compositions and methods are described in detail below.

a. Dithiocarbamate Metal Chelate

The term “dithiocarbamate metal chelate” as defined herein is a compound that has a heavy metal bonded to a dithiocarbamate thiolate anion. The mode of bonding can vary, which can include covalent and/or non-covalent (e.g., electrostatic, hydrogen bonding, dipole-dipole, dative bonding, etc.). The dithiocarbamate metal chelates disclosed in U.S. Pat. No. 6,548,540 and methods for preparing the same can be used herein, the teachings of which are incorporated by reference. In one aspect, the dithiocarbamate metal chelate has the formula I

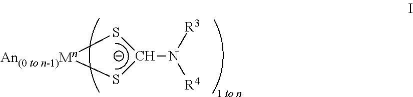

wherein R³ and R⁴ comprises, independently, hydrogen, or unsubstituted or substituted alkyl, alkenyl, aryl, alkoxy, or heteroaryl group; M is a heavy metal; An is a halide or an organic or inorganic pharmaceutically acceptable anion; and n is the valence of the metal. The term “heavy metal” as used herein includes any transition metal, lanthanide metal, or actinide metal, or group 5-8 non-metals in the periodic table. Examples of heavy metals useful herein include, but are not limited to, arsenic, bismuth, gallium, manganese, selenium, zinc, titanium, vanadium, chromium, iron, cobalt, nickel, copper, silver, and gold.

In one aspect, the dithiocarbamate metal chelates having the formula I can be synthesized either by treatment of a dithiocarbamate disulfide or the dithiocarbamate thiolate anion having the formula II with heavy metal salts. In one aspect, the dithiocarbamate thiolate anion that is the precursor to the dithiocarbamate metal chelate has the formula II

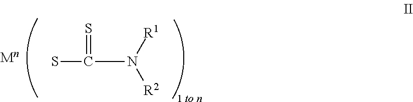

wherein R¹ and R² are, independently, hydrogen, or an unsubstituted or substituted alkyl, alkenyl, aryl, alkoxy, or heteroaryl group; M is an alkali metal comprising lithium, sodium, or potassium, or an alkali earth metal comprising calcium, magnesium, barium, and lithium; and n is the valence of the alkali metal or alkali earth metal. In one aspect, the compounds having the formula II can be produced by reacting a dithiocarbamate disulfide (e.g., a compound having the formula R¹R²N(S)CS—SC(S)NR¹R², where R¹ and R² are defined as above) with an alkali metal hydroxide or alkali earth metal hydroxide. Non-limiting examples of dithiocarbamates that can be oxidized to the corresponding dithiocarbamate disulfide include, but are not limited to, diethyldithiocarbamate, pyrrolodinedithiocarbamate, N-methyl, N-ethyl dithiocarbamates, hexamethylenedithiocarbamate, imidazolinedithiocarbamates, dibenzyldithiocarbamate, dimethylenedithiocarbamate, dipolyldithiocarbamate, dibutyldithiocarbamate, diamyldithiocarbamate, N-methyl, N-cyclopropylmethyldithiocarbamate, cyclohexylamyldithiocarbamate, pentamethylenedithiocarbamate, dihydroxyethyldithiocarbamate, N-methylglucosamine dithiocarbamate, and salts and derivatives thereof.

When a heavy metal salt is used to produce the dithiocarbamate metal chelate, the counterion of the heavy metal salt can be a variety of different anions. In one aspect, the anion of the heavy metal salt (i.e., An as shown in formula I), can be a halide (fluoride, chloride, bromide, iodide) or an organic or inorganic pharmaceutically acceptable anion. In other aspects, An is acetate, lactonate, glycinate, citrate, propionate or gluconate.

In one aspect, the dithiocarbamate metal chelate has the formula I, where R³ and R⁴ is an alkyl group such as, for example, an ethyl group. In another aspect, the dithiocarbamate metal chelate has the formula II, where R³ and R⁴ is an alkyl group such as, for example, an ethyl group, and M is copper or zinc. In a further aspect, the dithiocarbamate metal chelate has the formula I, where R³ and R⁴ is an alkyl group such as, for example, an ethyl group, M is copper, zinc, gold, or silver, and An is an organic anion such as, for example, acetate, lactonate, glycinate, citrate, propionate or gluconate.

b. Amphiphile

Amphiphiles useful herein are compounds possessing hydrophilic and lipophilic groups capable of forming micelles or liposomes. The amphiphiles should be biocompatible such that they possess minimal toxicity. Amphiphiles useful herein for preparing liposomes and micelles include homopolymers, copolymers, block-copolymers produced from biocompatible and biodegradable materials. Examples of such polymers include, but are not limited to, poly(amino acids); polylactides; poly(ethyleneimines); poly(dimethylaminoethylmethacrylates), copolymers of polyethyelene glycol and hydroxyalkyl acrylates and acrylamides (e.g., N-(2-hydroxypropyl)methacrylamide), PEG-β-poly(α-amino acids), poly(L-lactic acid)-poly(ethylene glycol) block copolymers, or poly(L-histidine)-poly(ethylene glycol) block copolymers.

In one aspect, the amphiphile is a poloxamer. In one aspect, the poloxamer is a nonionic triblock copolymer composed of a central hydrophobic chain of polyoxypropylene (e.g., (poly(propylene oxide)) flanked by two hydrophilic chains of polyoxyethylene (e.g., poly(ethylene oxide)). In one aspect, poloxamer has the formula

HO(C₂H₄O)_{b}(C₃H₆O)ₐ(C₂H₄O)_{b}OH

wherein a is from 10 to 100, 20 to 80, 25 to 70, or 25 to 70, or from 50 to 70; b is from 5 to 250, 10 to 225, 20 to 200, 50 to 200, 100 to 200, or 150 to 200. In another aspect, the poloxamer has a molecular weight from 2,000 to 15,000, 3,000 to 14,000, or 4,000 to 12,000. Poloxamers useful herein are sold under the tradename Pluronic® manufactured by BASF. Non-limiting examples of poloxamers useful herein include, but are not limited to, those in Table 1.

TABLE 1

| | | Average number | Average number | |
|---|---|---|---|---|
| Copolymer | MW | of EO units | of PO units | CMC (M) |
| F68 | 8,400 | 152.73 | 28.97 | 4.8 × 10⁻⁴ |
| P103 | 4,950 | 33.75 | 59.74 | 6.1 × 10⁻⁶ |
| P105 | 6,500 | 73.86 | 56.03 | 6.2 × 10⁻⁶ |
| P123 | 5,750 | 39.2 | 69.4 | 4.4 × 10⁻⁶ |
| F127 | 12,600 | 200.45 | 65.17 | 2.8 × 10⁻⁶ |
| L121 | 4,400 | 10.00 | 68.28 | 1.1 × 10⁻⁶ |

In other aspects, the amphiphile can be a lipid such as phospholipids, which are useful in preparing liposomes. Examples include phosphatidylethanolamine and phosphatidylcholine. In other aspects, the amphiphile includes cholesterol, a glycolipid, a fatty acid, bile acid, or a saponin.

c. Preparation of Compositions

The compositions described herein can readily prepared by mixing the dithiocarbamate metal chelate and amphiphile in the appropriate concentrations in a solvent to produce the micelle or liposome. In certain aspects, the dithiocarbamate thiolate anion and amphiphile are mixed in water followed by heating to produce micelles. The amount of dithiocarbamate metal chelate and amphiphile can vary. In one aspect, the amount of amphiphile should be sufficient such that the critical micelle concentration (CMC) is reached. The critical micelle concentration (CMC) is defined as the concentration of surfactants above which micelles are spontaneously formed. Table 1 provides the CMC of poloxamers useful herein as amphiphiles. In certain aspects, the concentration of amphiphile used can be several fold higher than the CMC of the amphiphile. It is contemplated that additional bioactive agents can be incorporated into the micelle or liposome in addition to the dithiocarbamate metal chelate. For example, other anticancer agents described below can be used herein in this aspect.

The compositions described herein are very stable. In other words, the dithiocarbamate metal chelate is protected from stringent physiological conditions such as reduced pH, and exposure to serum. Additionally, the compositions are easy to handle and can withstand purification steps such as filtration without the dithiocarbamate metal chelate leaching from the composition. Finally, the compositions are soluble in water. In general, the dithiocarbamate metal chelates useful herein are very insoluble in water, which prevents their administration by traditional techniques such as, for example, intravenous injection. Thus, when the dithiocarbamate thiolate anion is incorporated in a micelle or liposome, it is much easier to administer the dithiocarbamate metal chelate to the subject.

d. Pharmaceutical Compositions

In one aspect, any of the compositions described herein can be combined with at least one pharmaceutically-acceptable carrier to produce a pharmaceutical composition. The pharmaceutical compositions can be prepared using techniques known in the art. In one aspect, the composition is prepared by admixing the composition with a pharmaceutically-acceptable carrier. The term “admixing” is defined as mixing the two components together so that there is no chemical reaction or physical interaction. The term “admixing” also includes the chemical reaction or physical interaction between the compound having the formula I and the pharmaceutically-acceptable carrier.

Pharmaceutically-acceptable carriers are known to those skilled in the art. These most typically would be standard carriers for administration to humans, including solutions such as sterile water, saline, and buffered solutions at physiological pH.

Molecules intended for pharmaceutical delivery may be formulated in a pharmaceutical composition. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be parenterally, orally, subcutaneously, intralesionally, intraperitoneally, intravenously, or intramuscularly.

Preparations for administration include sterile aqueous or non-aqueous solutions, suspensions, and emulsions. Examples of non-aqueous carriers include alcoholic/aqueous solutions, emulsions or suspensions, and buffered media. Parenteral vehicles, if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

It will be appreciated that the actual preferred amounts of active compound in a specified case will vary according to the specific compound being utilized, the particular compositions formulated, the mode of application, and the particular situs and mammal being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (1999).

### II. Methods of Use

The compositions described herein are effective anticancer agents. Tumor cell resistance to chemotherapeutic agents represents a major problem in clinical oncology. In order to inhibit cell growth, induce cell differentiation, induce apoptosis, inhibit MDR phenotype, inhibit metastasis, inhibit angiogenesis or otherwise reverse or reduce the malignant phenotype of tumor cells using the methods and compositions of the present invention, a “target” cell is contacted with one or more compositions described herein. In certain aspects, the composition composed of the dithiocarbamate metal chelate and at least one other agent can be administered. The compositions described herein can improve the efficacy of chemo- and radiotherapy. One approach involves using the compositions described herein in combination with chemo- or radiotherapeutic intervention. This treatment option may offer a synergistic therapeutic effect along with the dithiocarbamate metal chelate. Different cancer therapeutic agents and methods of treatment utilizing such agents are well-known in the art.

In one aspect, the additional agent can be an anticancer agent. These compositions can be provided in a combined amount effective to kill or inhibit proliferation of the cell. This process may involve contacting the cells with the compositions and the agent(s) or factor(s) at the same time. This may be achieved by contacting the cell with a single composition or pharmacological formulation that includes both agents (e.g., dithiocarbamate metal chelate and chemotherapeutic agent), or by contacting the cell with two distinct compositions or formulations simultaneously, wherein one composition includes the dithiocarbamate metal chelate composition described herein and the other includes the agent.

Alternatively, any of the dithiocarbamate metal chelate composition treatments may precede or follow the other agent treatment by intervals ranging from minutes to weeks. In aspects where the other agent and any of the dithiocarbamate metal chelate compositions described herein are applied separately to the cell, a significant period of time should not expire between the time of each delivery, such that the agent and dithiocarbamate thiolate anion would still be able to exert an advantageously combined (e.g., synergistic) effect on the cell. In one aspect, the cell can be contacted with both modalities within about 12-24 h, or from about 6-12 h of each other, with a delay time of up to about 12 h. In some situations, it may be desirable to extend the duration of treatment with just the therapeutic agent, for example, where several days (2, 3, 4, 5, 6 or 7) to several weeks (1, 2, 3, 4, 5, 6, 7 or 8) lapse between the respective administrations.

Examples of anticancer agent include, but are not limited to, platinum compounds (e.g., cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, bendamustine), antitumor antibiotics (e.g., daunorubicin, doxorubicin, idarubicin, epirubicin, mitoxantrone, bleomycin, mytomycin C, plicamycin, dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, sunitinib), monoclonal antibodies (e.g., rituximab, cetuximab, panetumumab, tositumomab, trastuzumab, alemtuzumab, gemtuzumab ozogamicin, bevacizumab), nitrosoureas (e.g., carmustine, fotemustine, and lomustine), enzymes (e.g., L-Asparaginase), biological agents (e.g., interferons and interleukins), hexamethylmelamine, mitotane, angiogenesis inhibitors (e.g., thalidomide, lenalidomide), steroids (e.g., prednisone, dexamethasone, and prednisolone), hormonal agents (e.g., tamoxifen, raloxifene, leuprolide, bicalutamide, granisetron, flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, oxaprozin), selective cyclooxygenase-2 (COX-2) inhibitors, or any combination thereof.

In other aspects, the compositions described herein can be combined with therapies that induce DNA damage when applied to a cell. Such therapies include radiation such as, for example, γ-irradiation, X-ray, UV-irradiation, microwave, electronic emissions, and the like.

The methods described herein are applicable for treating a variety of different types of cancers. In one aspect, the cancer includes prostate, leukemia (e.g., acute myelogenous leukemia, acute promyelocytic, acute lymphoblastic leukemia, chronic myelogenous leukemia, chronic lymphocytic leukemia, hairy cell leukemia, plasma cell leukemia), myeloproliferative disorders (e.g., essential thrombocytosis, polythemia vera, primary myelofibrosis), myelodysplastic syndromes, lymphoma (Hodgkin and non-Hodgkin), testicular, head and neck, esophagus, stomach, liver, small intestine, gall bladder, rectum, anus, sarcoma, uterus, cervix, vulva, bladder, bone, renal, melanoma, colon, ovarian, lung, central nervous system, multiple myeloma, skin, or breast cancer.

In other aspects, the compositions described herein can be used as a purging agent. For example, stem cells can be collected from a patient afflicted with cancer (e.g., leukemia or multiple myeloma), and the stem cells can be treated with the compositions described herein to kill any residual malignant cells. This is also referred to herein as “purging” the graft. The treated stem cell can be subsequently used for a stem cell (bone marrow) transplant on the patient after high doses of chemotherapy/radiation.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

This example describes the synthesis of the copper salt of diethyldithiocarbamate (CuDETC). Equimolar amounts of copper gluconate (copper(II) D-gluconate, FW 453.84) and the sodium salt of diethyldithiocarbamate (sodium DETC, trihydrate, FW 225.31) were combined in water to synthesize the copper salt of DETC as follows. To a 50 cc polypropylene centrifuge tube containing 20 mL 0.1M NaDETC (0.451 gm/20 mL water), was added 10 mL of 0.2 M Cu gluconate (0.908 gm/10 mL water). The CuDETC product was collected by centrifugation for 15 mM at 2,400 rpm. The supernatant was discarded and the product was washed with distilled water (3×50 mL). The CuDETC product was then dried under flowing nitrogen at room temperature (22° C.). The chemicals were obtained from Sigma-Aldrich Chemical Co., St. Louis, Mo. A stock solution of the CuDETC product was prepared by dissolving 0.0217 gm of the powder in 8.0 mL of anhydrous dimethylsulfoxide in a glass vial. The approximate concentration at 0.0027 gm/mL was 10 mM (assuming a FW 265.8 for CuDETC).

### Example 2

This example describes the preparation of micelles loaded with CuDETC. Pluronic® 123 surfactant was obtained from BASF Corp., Mount Olive, N.J.). A stock solution of 20% w/w P123 was prepared by adding 180 gm of distilled water to 20.0 gm of P123 in a clean glass bottle and mixing to dissolve. Phosphate buffered saline (PBS), pH 7.4, was prepared by dissolving 58.4 gm NaCl (0.137 moles), 74.5 gm KCL (0.0027 moles), 142.0 gm Na₂HPO₄ (0.01 moles) and 136.1 g KH₂PO₄ (0.002 moles) in 1 L of distilled water and adjusting the final pH to 7.4. A stock solution of 2.0% P123 was prepared by adding 1.0 mL of 20% P123 stock solution to 9.0 mL of PBS. A stock solution of 0.5% P123 was prepared by adding 1.0 mL of 20% P123 stock solution to 39.0 mL of PBS.

For micelle preparation, a 400 micromolar CuDETC solution in 2.0% P123 was prepared in a glass vial by adding 0.006 mL of 10 mM CUDETC/DMSO stock to 1.44 mL of 2.0% P123 solution in PBS and gently mixing. Another micelle preparation was made using 64 micromolar CuDETC solution in 0.5% P123 in a glass vial by adding 0.01 mL of 10 mM CUDETC/DMSO stock to 1.55 mL of 0.5% P123 solution in PBS and gently mixing.

### Example 3

This example describes the preparation of micelles loaded with AgDETC. The silver salt of diethyldithiocarbamate (AgDETC, FW 265.14), was obtained from Sigma-Aldrich. A stock solution of AgDETC at 2 mM concentration was prepared by adding 0.0051 gm of AgDETC to 10.0 mL of anhydrous dimethylsulfoxide (DMSO) in a glass vial and heating to 60-70° C. to dissolve. Because AgDETC easily precipitated at room temperature this solution was used within 30-45 minutes to make a micelle solution.

For micelle preparation, a 60 micromolar AgDETC solution in 2.0% P123 was prepared in a glass vial by adding 0.02 mL of fresh 2 mM AgDETC/DMSO stock to 0.647 mL of 2.0% P123 solution in PBS and gently mixing.

### Example 4

Chelates of DETC and copper (Cu²⁺) or silver (Ag⁺) were synthesized. The in vitro anti-leukemic activity of both chelates using the HL-60 myeloid leukemia cell line was compared. For that purpose, the DETC/metal chelates were formulated in Pluronic® micelles using the P123 Pluronic® polymer. The DETC/metal chelates were initially dissolved in dimethylsulfoxide (DMSO). This 2 mM DETC/metal stock in DMSO was then mixed with a polymer stock and micellization was allowed to occur at room temperature. The final polymer concentration was 2% by weight. P123 polymer micelles loaded with DETC/metal were then added to HL-60 cells. DETC/metal chelates were added at concentrations of up to 0.24 μM. Cell growth was assessed after 3 days using the MTS assay. The final amount of DMSO in solution reached a maximum of 0.12%.

Cells treated with vehicle only showed decreased growth, likely due to the presence of DMSO. However, cells treated with either DETC/Cu²⁺ or DETC/Ag⁺ chelates suffered growth inhibition to a much greater extent. The DETC/Cu²⁺ chelate seemed to be more potent than the DETC/Ag⁺ chelate.

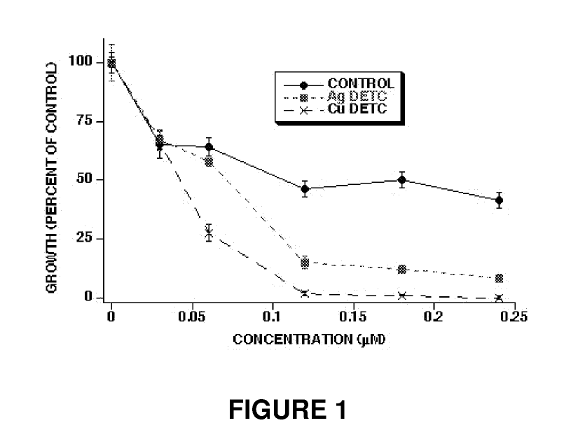

As shown in FIG. 1, HL-60 cells were cultured with the indicate concentrations of DETC/Cu or DETC/Ag chelate for 3 days upon which cell growth was measured using the MTS assay. The compounds were formulated in P123 Pluronic® micelles. The results show the potential efficacy of dithiocarbamate/metal chelates for the treatment of AML.

### Example 5

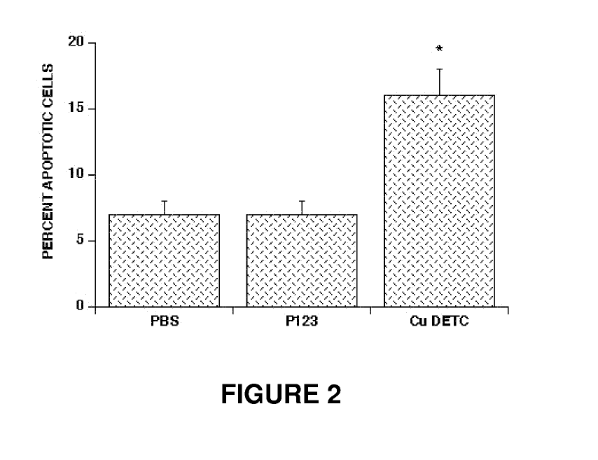

This example illustrates leukemia cell apoptosis induction by DETC/Cu. A DETC/Cu²⁺ chelate was synthesized and formulated as detailed above. HL-60 cells were treated with DETC/Cu²⁺ at a concentration of 0.32 μM for 24 hours upon which the number of apoptotic cells was determined using propidium iodide (PI) staining and flow cytometry. Analysis for the presence of apoptotic cells was modeled using the ModFit software. Cells treated with PBS or P123 vehicle control had 7±1% apoptotic cells each (FIG. 2; Averages and SEM of 3 separate experiments. * indicates P<0.05 as compared to PBS control). Cells treated with DETC/Cu²⁺ had 16±2% apoptotic cells.

### Example 6

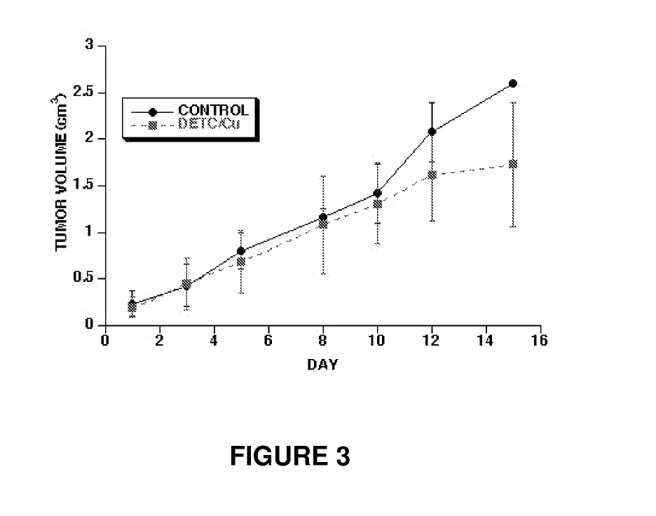

In another preliminary experiment, the effect of DETC/metal chelates on leukemia cell growth in vivo was studied. NOD/SCID IL2Rγ^{null} mice were implanted bilaterally in the flanks with 19×10⁶ HL-60 cells. When tumors became palpable, treatment was started with DETC/Cu formulate in P123 Pluronic® micelles and administered at a dose of 2 μmol/kg intravenously every other days. No obvious toxicity with this dose and schedule of administration was observed. There were 4 mice in each treatment group (Pluronic® P123 micelle control and DETC/Cu in P123 micelles). After 12 days of therapy, tumor volume curves started showing delay in tumor growth in DETC/Cu-treated mice as opposed to controls. The results are graphically shown in FIG. 3. The experiment was terminated on day 15. At that point 3 out of 4 control animals had died and the one remaining alive had tumor volumes that had exceeded 10% of initial body weight. None of the animals treated with DETC/Cu died. This experiment suggests that dithiocarbamate/metal copper have in vivo anti-leukemic activity.

### Example 7

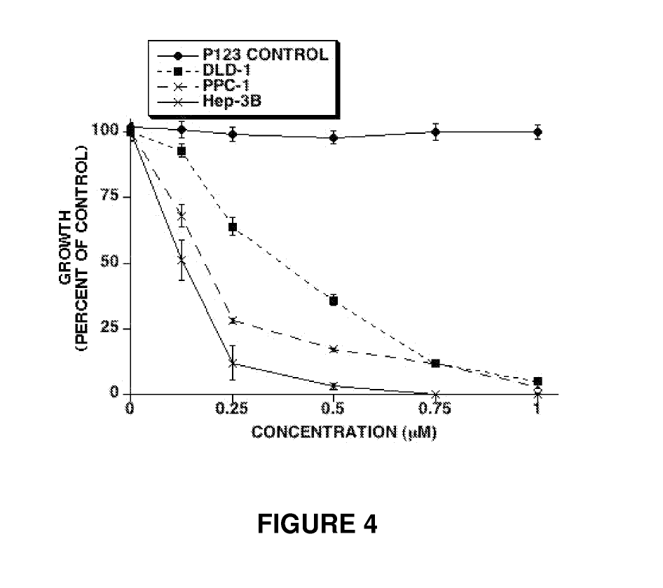

The effect of DETC/Cu²⁺ chelates on the growth of 3 solid tumor cell lines in vitro was studied. The cell lines studied were the colon cancer cell line DLD-1, the prostate cancer cell line PPC-1 and the hepatoma cell line Hep-3B. DETC/Cu²⁺ chelates were synthesized as previously described in Example 1. The DETC/Cu²⁺ chelates were solubilized in P123 Pluronic® micelles. Cells in logarithmic growth phase were cultured in RPMI1640/10% FBS media at a concentration of 120,000 cells/mL. DETC/Cu²⁺ was added to the cells at concentrations ranging from 0 to 1 μM. In parallel, cells were cultured with an equivalent volume of P123 control polymers. Two days after drug addition, cell growth was assayed using the CellTiter 96 assay from Promega (Madison, Wis.) using the manufacturer's protocol. Results show that DETC/Cu²⁺ inhibited the growth of 3 different solid tumor cell lines at submicromolar concentrations (FIG. 4). The 50% growth inhibitory concentration (IC₅₀) for DLD-1, PPC-1 and Hep-3B cells was 0.37, 0.18, and 0.13 μM, respectively. P123 control Pluronic® did not affect cell growth. Results shown in FIG. 4 are the average with standard error of the mean of triplicate experiments.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A composition produced by the process comprising admixing
(a) a dithiocarbamate metal chelate; and
(b) an amphiphile,
wherein the amount of amphiphile is sufficient to produce a liposome or micelle.

2. The composition of claim 1, wherein the dithiocarbamate metal chelate comprises the formula II
wherein R³ and R⁴ comprises, independently, hydrogen, or unsubstituted or substituted alkyl, alkenyl, aryl, alkoxy, or heteroaryl group; M is a transition metal; An is an anion comprising a halide or an organic or inorganic pharmaceutically acceptable anion; and n is the valence of the metal.

3. The composition of claim 2, wherein M comprises arsenic, bismuth, gallium, manganese, selenium, zinc, titanium, vanadium, chromium, iron, cobalt, nickel, copper, silver, silver, and gold; An is an anion comprising chloride, bromide, iodide, acetate, or an organic or inorganic pharmaceutically acceptable anion.

4. The composition of claim 2, wherein R³ and R⁴ comprise an alkyl group.

5. The composition of claim 2, wherein R³ and R⁴ comprise an ethyl group.

6. The composition of claim 2, wherein M is arsenic, bismuth, gallium, manganese, selenium, zinc, titanium, vanadium, chromium, iron, cobalt, nickel, copper, silver, and gold.

7. The composition of claim 2, wherein An is an organic anion comprising citrate, acetate, glyconate, glycinate, propionate, or lactate.

8. The composition of claim 1, wherein the amphiphile comprises a phospholipid, cholesterol, a glycolipid, a fatty acid, bile acid, or a saponin.

9. The composition of claim 1, wherein the amphiphile comprises poly(amino acids); polylactides; poly(ethyleneimines); poly(dimethylaminoethylmethacrylates), copolymers of polyethyelene glycol and hydroxyalkyl acrylates and acrylamides, PEG-β-poly(α-amino acids), poly(L-lactic acid)-poly(ethylene glycol) block copolymers, or poly(L-histidine)-poly(ethylene glycol) block copolymers.

10. The composition of claim 1, wherein the amphiphile comprises a poloxamer.

11. The composition of claim 10, wherein the poloxamer comprises a polyethylene oxide-polypropylene oxide-polyethylene oxide triblock copolymer.

12. The composition of claim 1, wherein the composition further comprises one or more anticancer agents.

13. The composition of claim 12, wherein the anticancer agent comprises a platinum compound, an alkylating agent, an antitumor antibiotic, an antimetabolite, a nucleoside analog, a topoisomerase inhibitor, a hypomethylating agent, a proteosome inhibitor, an epipodophyllotoxin, a vinca alkaloid, a tyrosine kinase inhibitor, a monoclonal antibody, a nitrosourea, an enzyme, a biological agent, a hexamethylmelamine, mitotane, an angiogenesis inhibitor, a steroid, a hormonal agent, an aromatase inhibitor, arsenic trioxide, tretinoin, a nonselective cyclooxygenase inhibitor, a selective cyclooxygenase-2 (COX-2) inhibitor, or any combination thereof.

14. The composition of claim 1, wherein the composition comprises a micelle.

15. The composition of claim 1, wherein one or more anticancer agents are admixed with components (a) and (b).

16. A pharmaceutical composition comprising the composition in claim 1 and a pharmaceutically acceptable carrier.

17. A method for treating cancer comprising administering to a subject the composition of claim 1.

18. A method for killing cancer cells comprising contacting the cancer cells with the composition of claim 1.

19. A method for preventing or reducing the growth of cancer cells comprising contacting the cancer cells with the composition of claim 1.

20. The method of claim 17, wherein the cancer comprises prostate, leukemia, a myeloproliferative disorder, a mylodysplastic syndrome, lymphoma, testicular, head and neck, esophagus, stomach, liver, small intestine, gall bladder, rectum, anus, sarcoma, uterus, vulva, cervix, bladder, bone, renal, melanoma, colon, ovarian, lung, central nervous system, multiple myeloma, skin cancer, or breast cancer.

21. The method of claim 17, wherein the composition is administered parenterally.

22. The method of claim 17, wherein the composition is administered parenterally, orally, subcutaneously, intralesionally, intraperitoneally, intravenously, or intramuscularly.

23. The method of claim 17, wherein the composition is used in combination with radiation therapy.
